(19) United States
(12) Patent Application Publication
(10) Pub. No.: US 2019/0126001 A1
(43) Pub. Date: May 2, 2019

(54) SOOTHING APPARATUS FOR PROMOTING INFANT SLEEP

(71) Applicant: NEPTUNE AZURE LIMITED, Hong Kong (CN)

(72) Inventor: Leonidas Kwong, Hong Kong (CN)

(21) Appl. No.: 15/867,780

(22) Filed: Jan. 11, 2018

(30) Foreign Application Priority Data
Nov. 2, 2017 (CN) 201711065450.9

(51) Int. Cl. A61M 21/02

(52) U.S. Cl. CPC A61M 21/02; A61M 2021/0027

(57) ABSTRACT

The present disclosure relates to the technical field of articles for infants, and particularly discloses a soothing apparatus for promoting infant sleep, including a first dioptric lens, a reflector assembly and an LED lamp successively from top to bottom, and further including: a rotating assembly located between the LED lamp and the reflector assembly. A plurality of light transmission pieces are arranged in the rotating assembly. Positions of the light transmission pieces are changed with rotation of the rotating assembly. Light rays emitted by the LED lamp penetrate through the rotating assembly and the light transmission pieces and then enter the first dioptric lens through reflection of the reflector assembly.

### TECHNICAL FIELD

The present application claims priority to Chinese Patent Application No. 201711065450.9 filed on Nov. 2, 2017, which is incorporated herein by reference in its entirety.

The present disclosure relates to the technical field of articles for infants, and particularly relates to a soothing apparatus for promoting infant sleep.

### BACKGROUND

At present, a plurality of devices for promoting infant sleep exist on a market. The devices are mainly classified into two categories: the first category is used for promoting infant sleep mainly by playing music such as ballad or cradlesong or the like; and the other category is used for promoting infant sleep mainly by projecting images such as dots or stars or the like on ceilings. A traditional device for promoting infant sleep is used for changing a projected image in a manner of supplying power for LED lamp beads of different colors. Therefore, only the single color image is projected, and those images do not have any motion.

### SUMMARY

A purpose of the present disclosure is to: provide a soothing apparatus for promoting infant sleep capable of projecting a moveable image and effectively prompting infants to enter a sleep state.

To achieve the purpose, the present disclosure provides a soothing apparatus for promoting infant sleep, including a first dioptric lens, a reflector assembly and an LED lamp successively from top to bottom, and further including:

a rotating assembly located between the LED lamp and the reflector assembly, wherein a plurality of light transmission pieces are arranged in the rotating assembly; positions of the light transmission pieces are changed with rotation of the rotating assembly; and light rays emitted by the LED lamp penetrate through the rotating assembly and the light transmission pieces and then enter the first dioptric lens through reflection of the reflector assembly.

As an embodiment, the rotating assembly includes:

a motor;

a rotating ring which rotates under drive of the motor;

an upper sealing plate made of light transmission material and installed on one end of the rotating ring near the reflector assembly; and

a middle separating plate made of light transmission material and located on one side of the upper sealing plate near the LED lamp, wherein the light transmission pieces are located in a space formed by the upper sealing plate and the middle separating plate.

As an embodiment, the soothing apparatus further includes:

a second dioptric lens located on one side of the middle separating plate near the LED lamp, and

a third dioptric lens located on one side of the second dioptric lens near the LED lamp.

As an embodiment, the soothing apparatus further includes:

a light reflecting cup located between the second dioptric lens and the third dioptric lens, wherein the third dioptric lens is fixed to one end of the light reflecting cup near the LED lamp.

As an embodiment, the reflector assembly includes a first reflector, a second reflector and a third reflector, and reflecting surfaces of the first reflector, the second reflector and the third reflector are encircled to form a triangular space; and

light rays emitted by the LED lamp are reflected for multiple times in the triangular space and then enter the first dioptric lens.

As an embodiment, the light transmission pieces are colorless and transparent light transmission bodies, and a color-changing light-emitting diode is arranged in the LED lamp.

As an embodiment, the light transmission pieces are colored light transmission bodies, and the LED lamp is a white light LED.

As an embodiment, the soothing apparatus includes a housing and the housing includes:

a base in which the rotating assembly is located; and

an upper cover hinged with the base, wherein the first dioptric lens is located in a space formed by the base and the upper cover.

As an embodiment, the soothing apparatus further includes:

a dioptric cover located on one side of the first dioptric lens near the upper cover and configured to refract light rays, which leave the first dioptric lens, to an inner side of the upper cover.

As an embodiment, the soothing apparatus further includes a sound playing apparatus.

The present disclosure has beneficial effects as follows: a soothing apparatus for promoting infant sleep is provided and the light transmission pieces are arranged in the rotating assembly which is rotatable, so that a moveable image is projected, thereby effectively prompting infants to enter a sleep state.

### BRIEF DESCRIPTION OF DRAWINGS

The present disclosure is further described below in detail according to drawings and embodiments.

FIG. 1 is a schematic diagram illustrating appearance of a soothing apparatus for promoting infant sleep provided in an embodiment;

FIG. 2 is a schematic diagram illustrating a base provided in an embodiment;

FIG. 3 is a schematic diagram illustrating position of a motor provided in an embodiment;

FIG. 4 is a schematic structural diagram illustrating a reflector assembly provided in an embodiment;

FIG. 5 is a schematic diagram illustrating position of a light transmission piece provided in an embodiment; and

FIG. 6 is a schematic structural diagram illustrating a rotating assembly provided in an embodiment.

In the figures:

1. Housing; 101. Upper cover; 102. Base;

2. Sound playing apparatus;

3. Dioptric cover;

4. First dioptric lens;

5. Reflector assembly; 501. First reflector; 502. Second reflector; 503. Third reflector;

6. Rotating assembly; 601. Motor; 602. Rotating ring; 603. Upper sealing plate; 604. Middle separating plate;

7. Light transmission piece;

8. Second dioptric lens;

9. Light reflecting cup;

10. Third dioptric lens;

11. LED lamp.

### DETAILED DESCRIPTION

The technical solution of the present disclosure will be further described below in combination with drawings through specific embodiments.

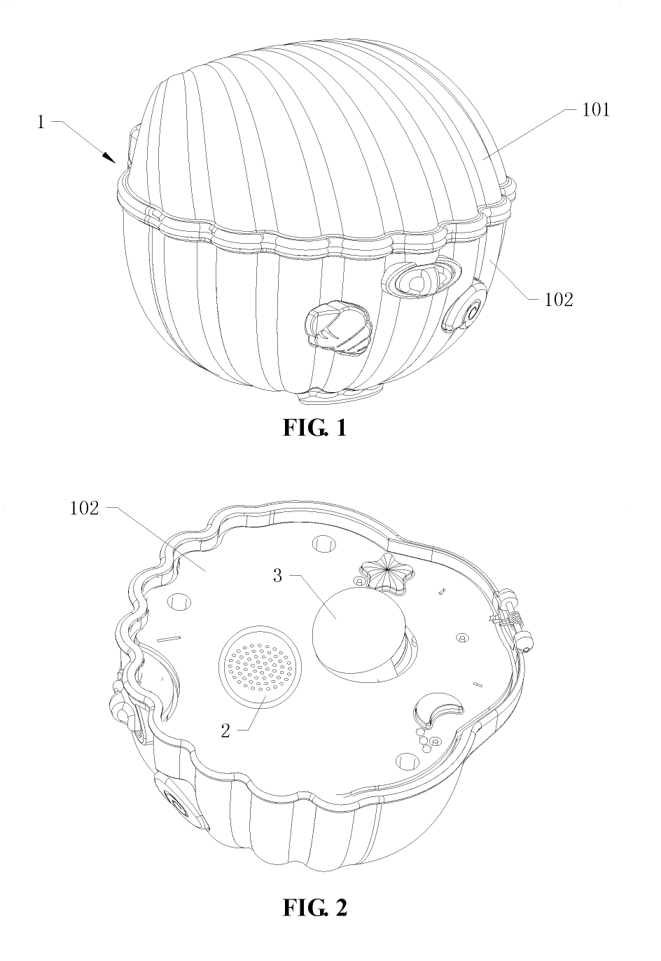

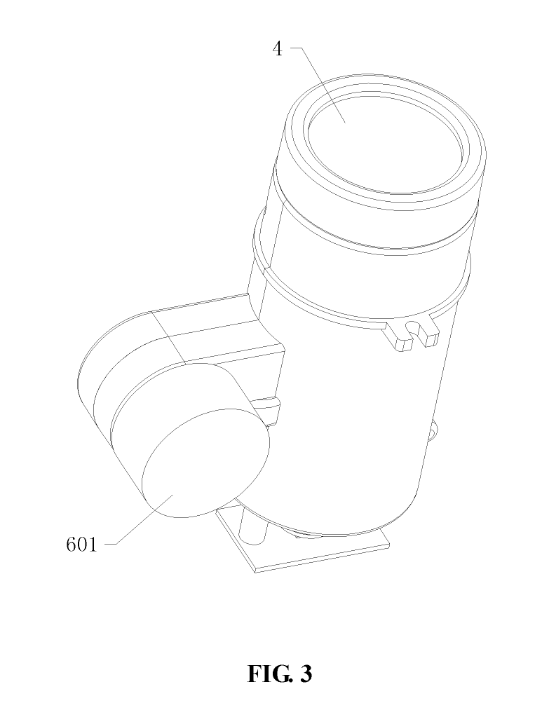

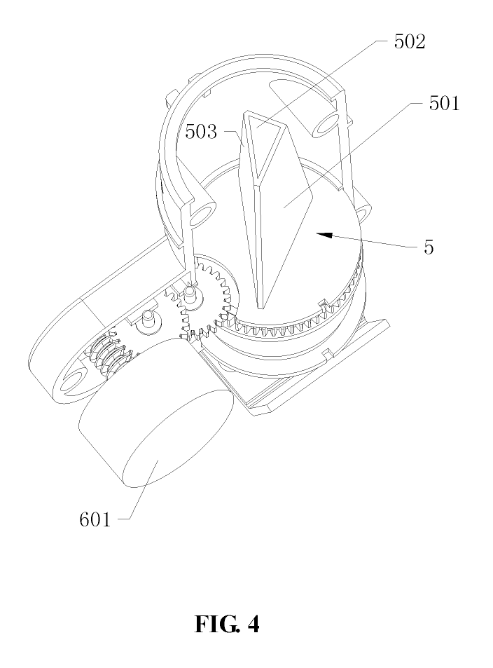

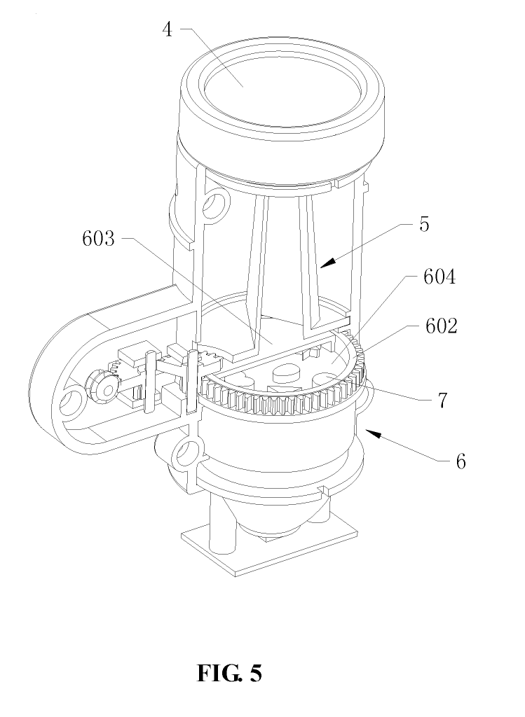

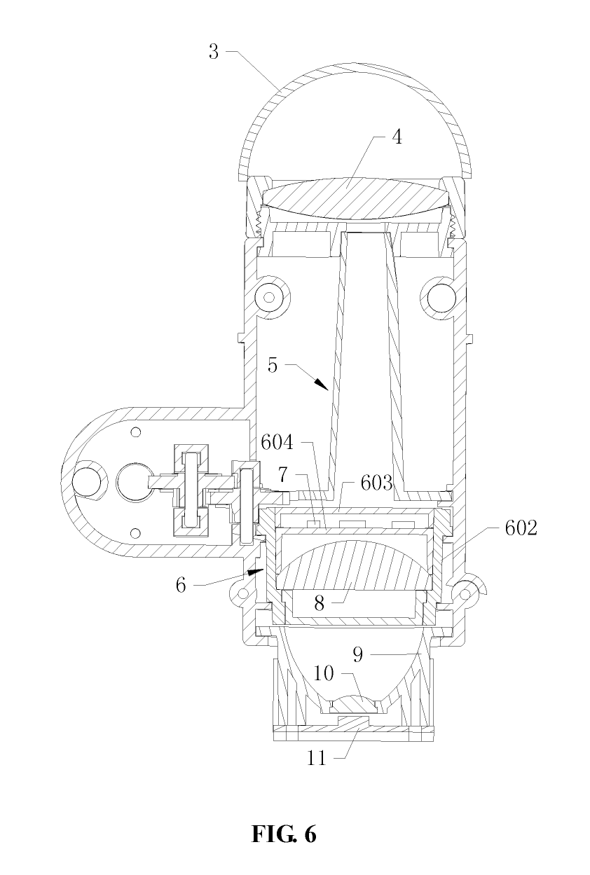

As shown in FIG. 1 to FIG. 6, the present disclosure discloses a soothing apparatus for promoting infant sleep, including a first dioptric lens 4, a reflector assembly 5 and an LED lamp 11 successively from top to bottom. The soothing apparatus for promoting infant sleep further includes a rotating assembly 6 located between the LED lamp 11 and the reflector assembly 5. A plurality of light transmission pieces 7 are arranged in the rotating assembly 6. Positions of the light transmission pieces 7 are changed with rotation of the rotating assembly 6. Light rays emitted by the LED lamp 11 penetrate through the rotating assembly 6 and the light transmission pieces 7 and then enter the first dioptric lens 4 through reflection of the reflector assembly 5. Specifically, a switch of the rotating assembly 6 is pressed, and then the rotating assembly 6 rotates. Since the light transmission pieces 7 are only placed in the rotating assembly 6 and not fixed in the rotating assembly 6, the light transmission pieces 7 perform irregular movement with the rotating assembly 6 when the rotating assembly 6 rotates. The light rays emitted by the LED lamp 11 successively pass through a bottom of the rotating assembly 6, the light transmission pieces 7 and a top of the rotating assembly 6, and then are changed with colors, positions, shapes and the like of the light transmission pieces 7. The changed light rays are projected outwards through the reflector assembly 5 and the first dioptric lens 4. For example, focusing can be performed by rotating the first dioptric lens 4, so as to project an image to a ceiling or wall, thereby obtaining various movable projected images.

In the present embodiment, the rotating assembly 6 includes a motor 601, a rotating ring 602, an upper sealing plate 603 and a middle separating plate 604. The rotating ring 602 rotates under drive of the motor 601. The upper sealing plate 603 is made of light transmission material, and is installed on one end of the rotating ring 602 near the reflector assembly 5. The middle separating plate 604 is made of light transmission material, and is located on one side of the upper sealing plate 603 near the LED lamp 11. The light transmission pieces 7 are located in a space formed by the upper sealing plate 603 and the middle separating plate 604. Specifically, an outer side of the rotating ring 602 is connected with the motor 601 through gears. When the motor 601 is operated, the motor 601 drives the rotating ring 602 to rotate and the rotating ring 602 drives the upper sealing plate 603 and the middle separating plate 604 to rotate.

In the present embodiment, the soothing apparatus for promoting infant sleep further includes a second dioptric lens 8 and a third dioptric lens 10. The second dioptric lens 8 is located on one side of the middle separating plate 604 near the LED lamp 11. The third dioptric lens 10 is located on one side of the second dioptric lens 8 near the LED lamp 11. Specifically, axes of the second dioptric lens 8 and the third dioptric lens 10 are on a same straight line. When the rotating ring 602 rotates, the second dioptric lens 8 also rotates with the rotating ring 602. The second dioptric lens 8 and the third dioptric lens 10 have effects as follows: light rays emitted from the LED lamp 11 with a point light source are irradiated on the middle separating plate 604 in parallel, i.e., after light rays emitted by the LED lamp 11 pass through the third dioptric lens 10 and the second dioptric lens 8, propagation directions of the light rays are changed to be perpendicular to the middle separating plate 604. Since the light rays penetrating through the light transmission pieces 7 are parallel light, a projected image is only amplified in an equal proportion compared with the light transmission pieces 7 and is not deformed.

In the present embodiment, the soothing apparatus for promoting infant sleep further includes a light reflecting cup 9. The light reflecting cup 9 is located between the second dioptric lens 8 and the third dioptric lens 10. The third dioptric lens 10 is fixed to one end of the light reflecting cup 9 near the LED lamp 11. Specifically, the light reflecting cup 9 can enhance light gathering capability.

In the present embodiment, the reflector assembly 5 includes a first reflector 501, a second reflector 502 and a third reflector 503. Reflecting surfaces of the first reflector 501, the second reflector 502 and the third reflector 503 are encircled to form a triangular space. Light rays emitted by the LED lamp 11 are reflected for multiple times in the triangular space and then enter the first dioptric lens 4.

In the present embodiment, the light transmission pieces 7 may be colorless and transparent light transmission bodies. A color-changing light-emitting diode is arranged in the LED lamp 11. Specifically, the light transmission pieces 7 may be starfishes, stars, cuboids, round balls and the like made of acrylic material. Color-changeable light rays project color-changeable images. Of course, alternatively, the light transmission pieces 7 are colored light transmission bodies, and the LED lamp 11 is a white light LED. Specifically, the light transmission pieces 7 may include red stars, blue balls, yellow triangular prisms and the like. After white light passes through the colored light transmission bodies, a projected image has a color.

In the present embodiment, the soothing apparatus for promoting infant sleep includes a housing 1. The housing 1 includes a base 102 and an upper cover 101. The rotating assembly 6 is located in the base 102. The upper cover 101 is hinged with the base 102. The first dioptric lens 4 is located in a space formed by the base 102 and the upper cover 101.

In the present embodiment, the soothing apparatus for promoting infant sleep further includes a dioptric cover 3. The dioptric cover 3 is located on one side of the first dioptric lens 4 near the upper cover 101 and is configured to refract light rays, which leave the first dioptric lens 4, to an inner side of the upper cover 101. The dioptric cover 3 may be sleeved on the first dioptric lens 4, and may also be taken out of the first dioptric lens 4. When image needs to be projected to the inner side of the upper cover 101, the dioptric cover 3 is sleeved; and when image needs to be projected to the ceiling or wall, the dioptric cover 3 is taken out.

In the present embodiment, the soothing apparatus for promoting infant sleep further includes a sound playing apparatus 2. Specifically, the sound playing apparatus 2 can play music and the like, and infant sleep is further promoted in combination with a changeable projection.

In the present embodiment, the upper sealing plate 603, the middle separating plate 604, the light transmission pieces 7 and the like can be made and obtained by glass, the acrylic material and the like.

The soothing apparatus for promoting infant sleep provided by the present disclosure has the advantages that: movable projected patterns are made; and the projected patterns are multicolored images, not single-color images. Harmonious light rays and patterns can make infants sleep better.

“First”, “second” and the like in the present disclosure are only intended to make a differentiation in description, and do not have special meanings.

It should also be noted that various specific technical features described in above specific embodiments can be combined in any appropriate mode in case of no conflict. To avoid unnecessary repetition, various possible combination modes are not separately illustrated in the present disclosure.

In addition, various embodiments of the present disclosure can also be arbitrarily combined, and shall also be regarded as the disclosure of the present disclosure as long as embodiments do not violate the idea of the present disclosure.

What is claimed is:

1. A soothing apparatus for promoting infant sleep, comprising a first dioptric lens, a reflector assembly and an LED lamp successively from top to bottom, and further comprising:
a rotating assembly located between the LED lamp and the reflector assembly, wherein a plurality of light transmission pieces are arranged in the rotating assembly; positions of the light transmission pieces are changed with rotation of the rotating assembly; and light rays emitted by the LED lamp penetrate through the rotating assembly and the light transmission pieces and then enter the first dioptric lens through reflection of the reflector assembly.

2. The soothing apparatus for promoting infant sleep according to claim 1, wherein the rotating assembly comprises:
a motor;
a rotating ring which rotates under drive of the motor;
an upper sealing plate made of light transmission material and installed on one end of the rotating ring near the reflector assembly; and
a middle separating plate made of light transmission material and located on one side of the upper sealing plate near the LED lamp, wherein the light transmission pieces are located in a space formed by the upper sealing plate and the middle separating plate.

3. The soothing apparatus for promoting infant sleep according to claim 2, further comprising:
a second dioptric lens located on one side of the middle separating plate near the LED lamp, and
a third dioptric lens located on one side of the second dioptric lens near the LED lamp.

4. The soothing apparatus for promoting infant sleep according to claim 3, further comprising:
a light reflecting cup located between the second dioptric lens and the third dioptric lens, wherein the third dioptric lens is fixed to one end of the light reflecting cup near the LED lamp.

5. The soothing apparatus for promoting infant sleep according to claim 1, wherein
the reflector assembly comprises a first reflector, a second reflector and a third reflector, and reflecting surfaces of the first reflector, the second reflector and the third reflector are encircled to form a triangular space; and
light rays emitted by the LED lamp are reflected for multiple times in the triangular space and then enter the first dioptric lens.

6. The soothing apparatus for promoting infant sleep according to claim 1, wherein the light transmission pieces are colorless and transparent light transmission bodies, and a color-changing light-emitting diode is arranged in the LED lamp.

7. The soothing apparatus for promoting infant sleep according to claim 1, wherein the light transmission pieces are colored light transmission bodies, and the LED lamp is a white light LED.

8. The soothing apparatus for promoting infant sleep according to claim 1, wherein the soothing apparatus comprises a housing and the housing comprises:
a base in which the rotating assembly is located; and
an upper cover hinged with the base, wherein the first dioptric lens is located in a space formed by the base and the upper cover.

9. The soothing apparatus for promoting infant sleep according to claim 8, further comprising:
a dioptric cover located on one side of the first dioptric lens near the upper cover and configured to refract light rays, which leave the first dioptric lens, to an inner side of the upper cover.

10. The soothing apparatus for promoting infant sleep according to claim 1, further comprising a sound playing apparatus.
